(19) United States
(12) Plant Patent Application Publication
(10) Pub. No.: US 2010/0293688 P1
(43) Pub. Date: Nov. 18, 2010

(54) ZOYSIAGRASS PLANT 'A-1'

(50) ;

(75) Inventor: Donald S. Loch, Alexandra Hills (AU)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: GeneGro Pty Ltd, Alexandra Hills (AU)

(21) Appl. No.: 12/453,550

(22) Filed: May 14, 2009

(51) Int. Cl. A01H 5/00

(52) U.S. Cl. PLT/390

(57) ABSTRACT

An asexually reproduced cultivar of perennial zoysiagrass that possesses a unique combination of characteristics including high turf quality and density under mowing, good shade tolerance, salinity tolerance, resistance to zoysia rust and Rhizoctonia blight, moderate to good resistance to tropical sod webworm and armyworm, and a distinctive DNA profile.

### BOTANICAL CLASSIFICATION

Zoysia matrella

### VARIETY DENOMINATION

‘A-1’

### BACKGROUND OF THE INVENTION

1. Field of the Invention

The present invention relates to a new and distinct asexually reproduced perennial zoysiagrass cultivar named ‘A-1’.

2. Description of Prior Art

Zoysiagrasses are a widely used group of warm-season turfgrasses in the southern United States. The group includes three species and their interspecific hybrids: Zoysia japonica Steud., Z. matrella (L.) Merr., and (rarely) Zoysia pacifica (Goudswaard) Hotta & Kuroki (formerly assigned to Z. tenuifolia Thiele). All are rhizomatous and stoloniferous, mat-forming perennials adapted to a wide range of edaphic conditions.

Compared with other warm-season turfgrasses such as Bermudagrasses and St. Augustinegrass, zoysiagrasses are very resistant to wear damage, but slow to spread laterally by stolons and rhizomes and are therefore slower to recover from wear damage. Z. japonica produces coarse to medium-textured leaves and is adapted from subtropical to cool temperate conditions, while Z. matrella produces medium to fine-textured leaves and is adapted to warmer climates from tropical through to warm temperate.

Prior art Z. matrella and Z. matrella×Z. japonica zoysiagrasses include ‘Diamond’ (U.S. Plant Pat. No. 10,636), ‘Cavalier’ (U.S. Plant Pat. No. 10,778), ‘Zorro’ (U.S. Plant Pat. No. 14,130), and ‘Royal’ (U.S. Plant Pat. No. 14,395).

### BRIEF SUMMARY OF THE INVENTION

The present invention relates to a new and distinct perennial Zoysia matrella zoysiagrass cultivar identified as ‘A-1’.

‘A-1’ differs from other known Z. matrella and Z. matrella×Z. japonica cultivars with respect to a number of morphological characteristics, shows greater winter hardiness, and has a distinctive DNA profile. ‘A-1’ produces shorter, narrower leaves (i.e., finer-textured foliage), shorter, erect tillers, and larger inflorescences on longer, thinner peduncles than ‘Cavalier’ and ‘Zorro’. Compared with ‘Royal’, ‘A-1’ has narrower leaves, but produces larger inflorescences on longer, thinner peduncles. ‘A-1’ produces longer stolon internodes, longer vertical tillers with more elongated leaves (i.e., greater length:width ratio), and larger inflorescences on longer, thicker peduncles than ‘Diamond’. ‘A-1’ also has good shade and salinity tolerance, is tolerant of zoysia rust and resistant to Rhizoctonia blight, and shows moderate to good resistance to tropical sod webworm and armyworm.

### BRIEF DESCRIPTION OF THE DRAWINGS

FIG. 1 is a photograph showing stolon comparisons of five Zoysia matrella and Z. matrella×Z. japonica cultivars: ‘A-1’, ‘Cavalier’, ‘Zorro’, ‘Royal’, and ‘Diamond’.

FIGS. 2A-2C are photographs of compound stolon nodes from ‘A-1’ showing a compressed node subtended by 3 stolon leaves (FIG. 2A), the progressive development of branching at nodes along a stolon (FIG. 2B), and complex branching from older compound nodes (FIG. 2C). Vestigal leaf blades are present on the stolon leaves.

FIG. 3 shows the dendrogram for ‘Royal’, ‘Zorro’, ‘Cavalier’, ‘A-1’, and ‘Diamond’ constructed from 71 markers generated from two ISSR primers using Jaccard's coefficient.

FIG. 4 presents the electropherogram for ‘A-1’ versus four other zoysiagrass genotypes using primer 6FAM (AG)₉C. The electropherograms (from the top panel to the bottom panel) are from ‘Royal’, ‘Zorro’, ‘Cavalier’, ‘A-1’, and ‘Diamond’.

### DETAILED DESCRIPTION OF THE INVENTION

‘A-1’ was selected from a breeding population of forty seedling Zoysia matrella plants from various parts of Southeast Asia (Japan, Philippines, China, Korea, Vietnam and Thailand). The original plants were vegetatively propagated and evaluated first in pots. Vegetative propagation was performed by taking stolons from the original plant and dividing them into stolon tip and single-node cuttings. These cuttings were placed into soilless peat-vermiculite potting mix in nursery cell trays to produce roots and grow through the potting medium before transplanting into larger pots.

A shortlist of selected genotypes was expanded to field plantings at Sheldon, QLD (Australia). The plants were vegetatively propagated in nursery cell trays prior to planting in the field. Once in the field, the plants were evaluated against existing Z. matrella and Z. matrella×Z. japonica hybrid cultivars under mowing heights from 10 to 25 mm and under shade levels ranging from 0 to 80%. ‘A-1’ from Okinawa (Japan) showed higher tiller density and a more prostrate growth habit than the parent ecotypes, and was selected from the wider breeding population on the basis of its superior turf colour, quality under mowing for 6 years, and its shade tolerance as shown by its ability to maintain density of the mown sward under greatly reduced light levels (70-80% shade). Additional observations regarding climatic adaptation were made in Cairns, QLD (Australia) and Melbourne, VIC (Australia) using vegetative propagules as described above. ‘A-1’ has remained true-to-type with no discernible off-types through more than four cycles of vegetative multiplication.

### DETAILED BOTANICAL DESCRIPTION

‘A-1’ is a perennial diploid (2n=40 chromosomes) zoysiagrass with a creeping growth habit. It spreads laterally by stolons and rhizomes, which produce short, erect tillers.

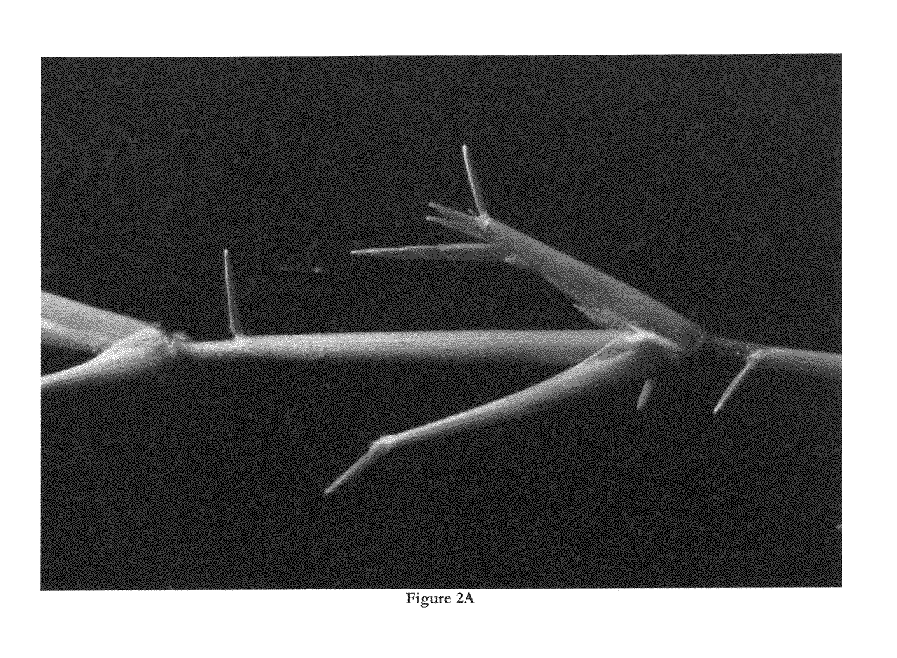

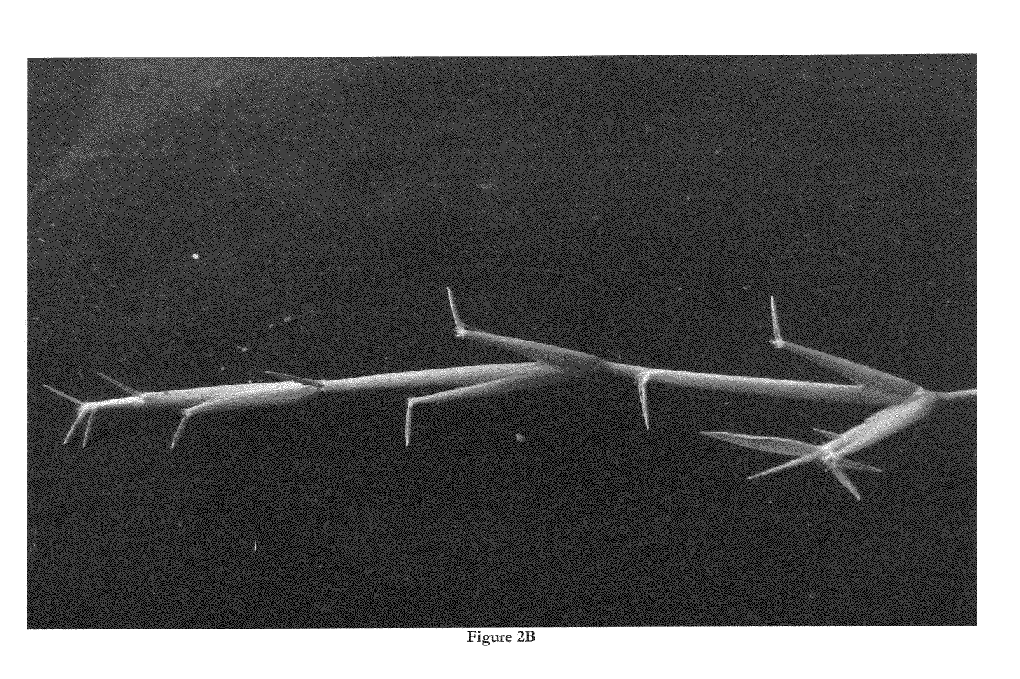

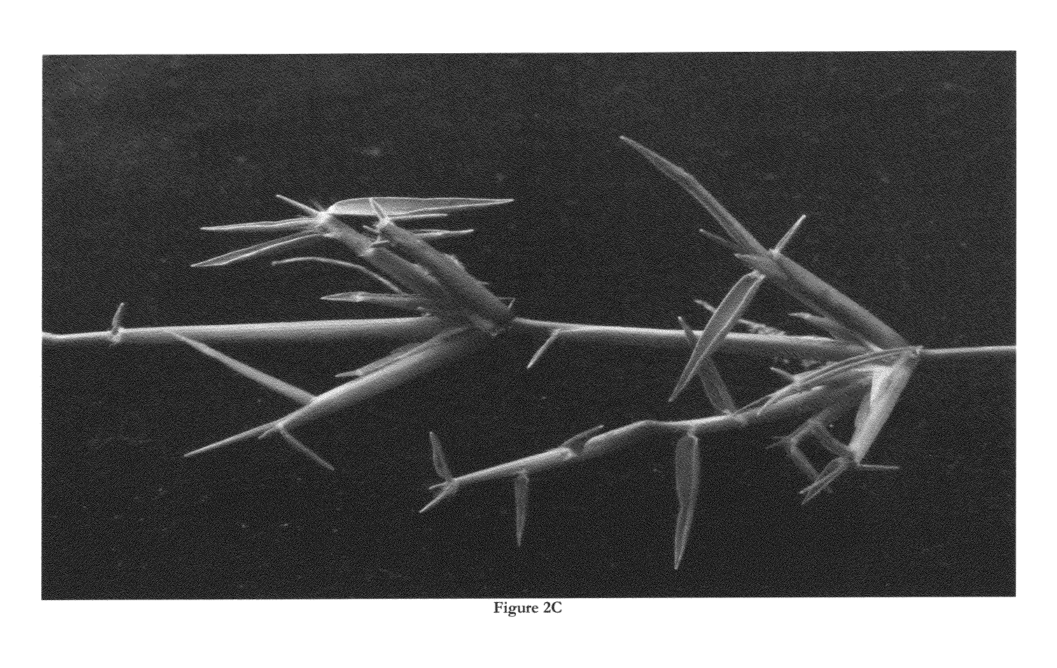

The stolons of ‘A-1’ consist of short, thin internodes separated by compound nodes, each of which is subtended by 3 leaves (FIG. 2). Structurally, each compound node consists of two compressed internodes and one normal internode and so can produce axillary stolon shoots (i.e., branches) from any or all of the three component nodes, starting from the component node furthest from the stolon tip and subtended by the outermost stolon leaf (FIG. 2).

Internodes on ‘A-1’ are longer than on ‘Diamond’, but comparable in length to those of ‘Cavalier’, ‘Zorro’ and ‘Royal’. ‘A-1’ produces fewer early stolon shoots per node than ‘Diamond’, ‘Cavalier’ and ‘Zorro’. Stolon color is reddish-purple (RHS N79A) when exposed to sunlight. Stolon leaf blades are greatly reduced (vestigal) compared with tiller leaves. Stolon leaf blades on ‘A-1’ are longer and wider (i.e., better developed) than those of ‘Cavalier’ ‘Zorro’, ‘Royal’ and ‘Diamond’.

Tiller length (i.e., vertical extension) on ‘A-1’ is shorter than ‘Cavalier’ and ‘Zorro’, but longer than ‘Diamond’. Leaf blades are rolled in the bud and later emerge to become flat and stiff and linear-triangular in shape. ‘A-1’ produces shorter, narrower leaf blades than ‘Cavalier’ and ‘Zorro’. Hairs are absent on both the abaxial and adaxial surfaces of the leaf blade and on the leaf sheath. Leaf blade color is dark green (RHS 137A—2001 edition). The ligule is a row of short silky hairs c. 2 mm long.

‘A-1’ flowers from about April to October in the southern hemisphere, and October to April in the northern hemisphere. The inflorescence is a short, spike-like raceme. ‘A-1’ produces longer peduncles and racemes and has more spikelets per inflorescence than ‘Cavalier’, ‘Zorro’, ‘Royal’, and ‘Diamond’. Peduncles on ‘A-1’ are thinner than those on ‘Cavalier’, ‘Zorro’, and ‘Royal’, but thicker than ‘Diamond’ peduncles.

‘A-1’ was compared against other Zoysia matrella and Z. matrella×Z. japonica cultivars ‘Diamond’, ‘Cavalier’, ‘Royal’, and ‘Zorro’ in a spaced-plant field trial at Cleveland, QLD (Australia) (Latitude 27°32′S., 153°15′E., elevation c. 50 masl). Morphological grouping characteristics used to select the most similar comparator varieties of common knowledge were stolon internode length, leaf blade length and width, leaf length and width on flowering tillers, peduncle length and width, and inflorescence length (Table 1).

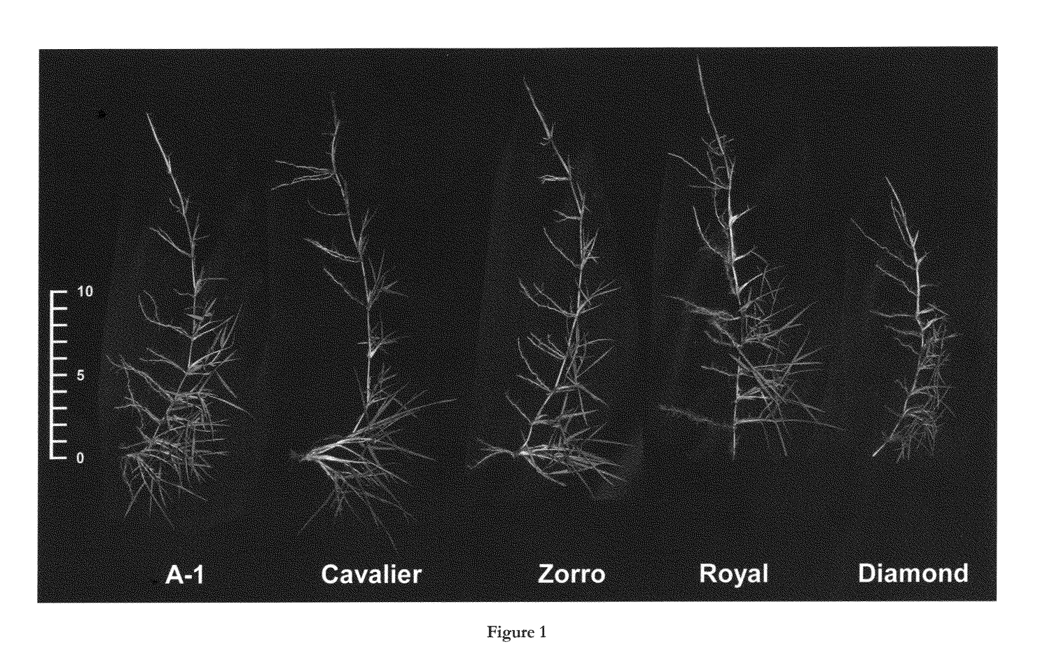

Rooted vegetative plugs 5 cm in diameter were taken from nursery stock and planted on a basaltic red ferrosol soil on Mar. 3, 2003 on a 1 m×1 m grid. Thirty spaced plants from each of the five cultivars were arranged in three randomized blocks with ten plants per plot. Weed control was achieved by a pre-emergence application of oxadiazon (repeated on Jul. 23, 2003) and with post-emergence fluroxypyr for broadleaf weeds on Mar. 23, 2003. Good nutrition was maintained by regular applications of slow release complete NPK fertilizer at one- to two-month intervals. The spaced plants were allowed to grow and develop without any mowing. Leaf and stolon colors were determined on Jul. 16, 2003. Diameter of spread was taken from four measurements per plant made on Aug. 22, 2003. Shoot and inflorescence characteristics were measured on two mature tillers between Sep. 17 and 19, 2003. Stolon stem and leaf characteristics were measured on two stolons per plant between Oct. 6 and 10, 2003. Digital images of stolon characteristics (FIG. 1) were taken Dec. 10, 2003.

TABLE 1
Morphological/Agronomic Data from Comparative Growing Trial

| | | | | | | LSD |
|---|---|---|---|---|---|---|
| | | ‘Cava- | | | ‘Dia- | (P = |
| Attribute | ‘A-1’ | lier’ | ‘Zorro’ | ‘Royal’ | mond’ | 0.05) |
| Mean plant diameter | 55.9 | 82.6 | 74.0 | 62.2 | 37.5 | 12.9 |
| after 173 days (cm) | | | | | | |
| Number of axillary | 2.30 | 3.18 | 3.05 | 2.83 | 4.13 | 0.51 |
| shoots at 4th stolon | | | | | | |
| node (spaced plants) | | | | | | |
| Length of 4th | 23.0 | 24.6 | 26.1 | 22.2 | 11.1 | 3.9 |
| internode from | | | | | | |
| stolon tip (mm) | | | | | | |
| Diameter of 4th | 1.37 | 1.39 | 1.36 | 1.44 | 1.36 | 0.11 |
| internode from | | | | | | |
| stolon tip (mm) | | | | | | |
| Length of outermost | 9.6 | 8.9 | 9.3 | 10.3 | 7.3 | 1.3 |
| leaf sheath on 4th | | | | | | |
| visible node from | | | | | | |
| stolon tip (mm) | | | | | | |
| Length of innermost | 20.1 | 18.1 | 17.4 | 19.1 | 13.5 | 2.7 |
| leaf sheath on 4th | | | | | | |
| visible node from | | | | | | |
| stolon tip (mm) | | | | | | |
| Length of leaf blade | 4.29 | 3.08 | 3.03 | 2.60 | 1.53 | 0.51 |
| on 4th visible node | | | | | | |
| from stolon tip (mm) | | | | | | |
| Width of leaf blade | 0.85 | 0.73 | 0.71 | 0.63 | 0.50 | 0.11 |
| on 4th visible node | | | | | | |
| from stolon tip (mm) | | | | | | |
| Length:width ratio | 5.07 | 4.26 | 4.27 | 4.17 | 3.07 | 0.64 |
| of leaf blade on | | | | | | |
| fourth visible node | | | | | | |
| from stolon tip | | | | | | |
| Length of flag leaf | 22.7 | 19.7 | 20.4 | 19.6 | 14.0 | 2.3 |
| sheath on flowering | | | | | | |
| tillers (mm) | | | | | | |
| Length of flag leaf | 3.90 | 3.61 | 3.80 | 3.21 | 3.35 | 0.66 |
| blade on flowering | | | | | | |
| tillers (mm) | | | | | | |
| Width of flag leaf | 0.71 | 0.82 | 0.67 | 0.68 | 0.66 | 0.16 |
| blade on flowering | | | | | | |
| tillers (mm) | | | | | | |
| Length:width ratio | 5.49 | 4.45 | 5.75 | 4.78 | 5.18 | 0.99 |
| of flag leaf blade on | | | | | | |
| flowering tillers | | | | | | |
| Length of sheath on | 10.22 | 14.10 | 16.07 | 12.55 | 7.96 | 1.30 |
| fourth leaf on | | | | | | |
| flowering tillers | | | | | | |
| (mm) | | | | | | |
| Length of blade on | 18.0 | 29.2 | 32.6 | 21.1 | 15.6 | 3.9 |
| fourth leaf on | | | | | | |
| flowering tillers | | | | | | |
| (mm) | | | | | | |
| Width of blade on | 1.61 | 1.93 | 2.01 | 1.66 | 1.53 | 0.27 |
| fourth leaf on | | | | | | |
| flowering tillers | | | | | | |
| (mm) | | | | | | |
| Length:width ratio | 11.12 | 15.04 | 16.16 | 12.83 | 10.33 | 1.03 |
| of fourth leaf blade | | | | | | |
| on flowering tillers | | | | | | |
| Length of peduncle | 41.5 | 34.9 | 35.1 | 32.3 | 19.7 | 5.8 |
| (mm) | | | | | | |
| Diameter of | 0.56 | 0.73 | 0.70 | 0.65 | 0.43 | 0.05 |
| peduncle (mm) | | | | | | |
| Mean spike length | 17.9 | 15.1 | 14.7 | 14.9 | 10.7 | 0.9 |
| (mm) | | | | | | |
| Number of | 23.5 | 18.0 | 17.3 | 16.3 | 9.0 | 1.8 |
| spikelets per | | | | | | |
| inflorescence | | | | | | |
| Color of stolon | N79A | N79A | N79A | N79A | N79A | — |
| stem exposed to | | | | | | |
| sunlight | | | | | | |
| Color of leaf blade | 137A | 137A | 137C | 137A | 137A | — |
| (RHS Colour | | | | | | |
| Chart, 2001 | | | | | | |
| edition) | | | | | | |

### STRESS RESISTANCE

‘A-1’ shows excellent salt tolerance. In a greenhouse experiment, six salinity levels covering the range from 60 to 25,600 ppm Total Dissolved Salts (TDS) applied as NaCl were imposed hydroponically through the irrigation water. After being held at the designated treatment levels for thirteen weeks, the level of leaf firing induced in ‘A-1’ was comparable to that in ‘Diamond’, ‘Cavalier’, ‘Zorro’, and ‘Royal’ (Table 2). The relative dry matter yield of clippings over the ten- to fourteen-week period in ‘A-1’ was lower than ‘Diamond’ and ‘Royal’ at the highest salinity level (25,600 ppm TDS), but was superior to the other four cultivars at 5,120 ppm TDS and generally comparable to them at intermediate salinity levels (Table 3).

TABLE 2
Effect of salinity level on percent leaf firing of Zoysia matrella
cultivars) after thirteen weeks of treatment. LSD (P = 0.05) = 10.3.

| | TDS (ppm) | | | | | |
|---|---|---|---|---|---|---|
| Cultivar | 60 | 5,120 | 10,240 | 15,360 | 20,480 | 25,600 |
| ‘A-1’ | 5.0 | 23.3 | 31.7 | 64.2 | 61.7 | 90.7 |
| ‘Diamond’ | 2.8 | 11.7 | 24.2 | 46.7 | 61.7 | 87.5 |
| ‘Cavalier’ | 4.5 | 30.8 | 40.0 | 69.2 | 72.5 | 94.7 |
| ‘Zorro’ | 6.7 | 22.5 | 45.8 | 72.5 | 71.7 | 91.7 |
| ‘Royal’ | 8.3 | 50.0 | 50.0 | 73.3 | 73.3 | 90.7 |

TABLE 3
Effect of salinity level on dried clipping yield of Zoysia
matrella cultivars relative to the control treatment (=1.000)
after fourteen weeks of treatment. LSD (P = 0.05) = 0.149.

| | TDS (ppm) | | | | | |
|---|---|---|---|---|---|---|
| Cultivar | 60 | 5,120 | 10,240 | 15,360 | 20,480 | 25,600 |
| ‘A-1’ | 1.000 | 1.073 | 0.773 | 0.448 | 0.270 | 0.081 |
| ‘Diamond’ | 1.000 | 0.680 | 0.604 | 0.427 | 0.449 | 0.307 |
| ‘Cavalier’ | 1.000 | 0.656 | 0.450 | 0.321 | 0.252 | 0.000 |
| ‘Zorro’ | 1.000 | 0.822 | 0.671 | 0.321 | 0.141 | 0.096 |
| ‘Royal’ | 1.000 | 0.716 | 0.833 | 0.462 | 0.326 | 0.238 |

‘A-1’ is tolerant of zoysia rust (Puccinia zoysiae) and is resistant to Rhizoctonia blight. It is also resistant to sod webworm (Herpetogramma licarsisalis) and armyworm (Pseudaletia spp., Spodoptera spp.), except where excessive nitrogen fertilizer use causes softer leaves.

‘A-1’ has shown superior winter hardiness to ‘Diamond’, ‘Cavalier’, ‘Zorro’ and ‘Royal’ in Melbourne, VIC (Australia). In trial plots at South Oakleigh (37°55′S., 145°06′E.), ‘A-1’ grew in faster from cells with rooted stolon cuttings and has maintained good ground cover and turf quality, while the other four cultivars showed poor winter survival and declined rapidly to very low levels of cover and quality.

### DNA PROFILING

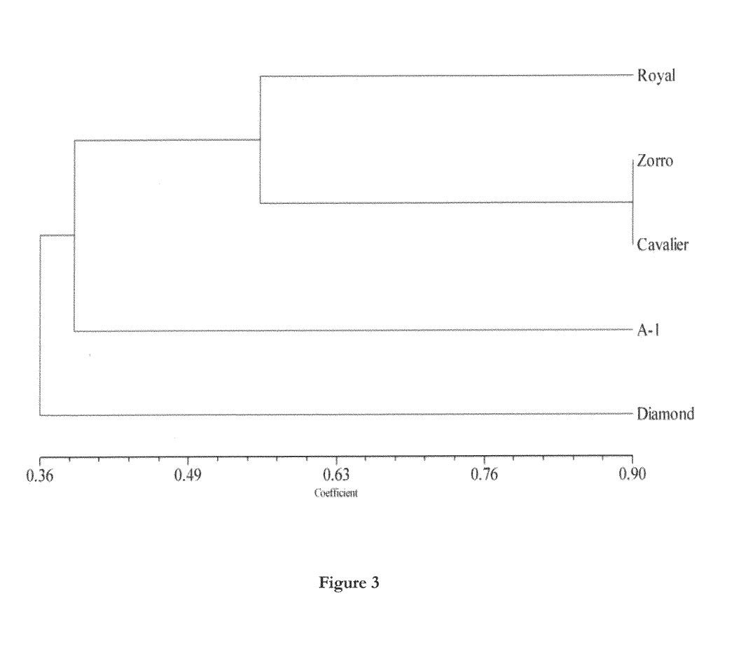

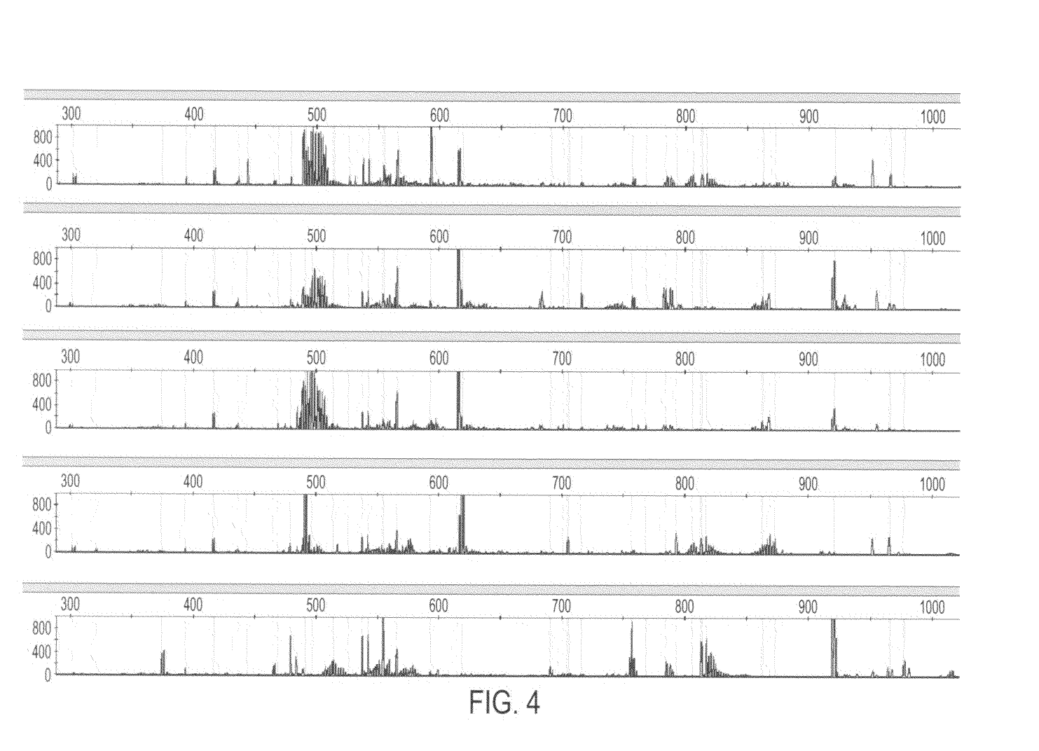

DNA was extracted from ground leaf material using a modified CTAB (cetyl tri-methyl ammonium bromide) procedure. Inter-Simple-Sequence-Repeat (ISSR) markers were generated by the polymerase chain reaction (PCR) using a GeneWorks thermal cycler and two fluorescently labelled primers, 6FAM (AG)₉C and NED (GA)₉T. Amplification products were separated by capillary electrophoresis using an ABI 3130 genotyper and visualised using GENEMAPPER® software. The dominant markers generated with both primers were then used to produce a dendrogram (FIG. 3) using pattern analysis. Distinctive marker loci were identified by both primers. FIG. 4, as an example, illustrates the distinctive marker loci identified using primer 6FAM (AG)₉C.

What is claimed is:

1. A new and distinct cultivar of Zoysia matrella Merr. plant named ‘A-1’ as described and illustrated herein.
